(19) United States
(12) Patent Application Publication
(10) Pub. No.: US 2025/0064705 A1
(43) Pub. Date: Feb. 27, 2025

(54) COMPOSITION FOR TINTED ADHESIVE SPRAY

(71) Applicant: EBIN NEW YORK, INC., South Hackensack, NJ (US)

(72) Inventor: Qing Zhang, Guangzhou (CN)

(21) Appl. No.: 18/806,696

(22) Filed: Aug. 16, 2024

Related U.S. Application Data
(60) Provisional application No. 63/536,798, filed on Sep. 6, 2023.

(30) Foreign Application Priority Data
Aug. 25, 2023 (CN) 2023110844119

(51) Int. Cl. A61K 8/55; A61K 8/19; A61K 8/81; A61Q 5/06

(52) U.S. Cl. CPC A61K 8/55; A61K 8/19; A61K 8/8152; A61Q 5/065

(57) ABSTRACT

The present invention related to a method for preparing a hair cosmetic composition. The method includes treating a colorant with a phytic acid; preparing a mixture including the colorant treated with phytic acid, and a solvent; grinding the mixture about 3 to 5 times using an ultra-precision colloid grinding machine; adding an adhesive component to the mixture; and adding a propellant to the mixture.

### CROSS-REFERENCE TO RELATED APPLICATION

This application relates to the subject matter disclosed in Chinese Patent Application No. 2023110844119 entitled “COMPOSITION FOR TINTED ADHESIVE SPRAY” and filed Aug. 25, 2023, the entire disclosure of which application is incorporated herein by reference. A copy of the aforesaid Chinese patent application is attached hereto as Appendix A and made part hereof.

### FIELD OF THE INVENTION

The present invention relates to a composition for a tinted adhesive spray, and, more particularly, a tinted adhesive spraying for affixing hair pieces and a method of making the same.

### BACKGROUND OF THE INVENTION

Hair cosmetic products with adhesive properties can be used to attach hair accessories such as hair extensions, wigs, or other hair products. However, the adhesive hair cosmetic products do not have coloring capabilities, which may be desirable to blend or hide seams or laces of the hair extensions or wigs. The adhesive hair cosmetic products are unable to incorporate coloring agents such as iron oxides because iron oxides are not soluble with mixtures containing the adhesive components. The lack of solubility can cause the iron oxides to be separated from the other components of the hair products and the iron oxides may not be evenly distributed throughout the composition of the hair product. Accordingly, the hair cosmetic compositions described herein and methods of making the same provide a stable composition containing adhesive and coloring agents.

### SUMMARY OF THE INVENTION

In one embodiment, the hair cosmetic composition includes iron-based colorants, a solvent, and adhesive components. In one embodiment, hair cosmetic composition is formed by placing the iron-based colorants, the solvent, the adhesive components, and a propellant into a pressure container to form an aerosol composition using a process generally understood in the art. In one embodiment, the propellant includes hydrofluorocarbon (HFC) 152a, HFC 134a, and dimethyl ether.

In one embodiment, the surface of the iron-based colorant is treated with phytic acid (PHY) to form a PHY-treated iron-based colorant. The iron-based colorant is treated with PHY to form a chelate, so that the surface of the toner has multiple hydrophilic hydroxyl groups (—OH). The multiple hydroxyl groups allow the iron-based colorant to disperse in a liquid phase more easily (e.g., a water phase). PHY is a natural hydrophilic surface treatment agent, which is suitable for water-based makeup and skin care products. The PHY surface treatment allows the iron-based colorant to have high toner loading capacity for easy toning and that the hydrophilicity and viscosity are not affected by pH. In one embodiment, the iron oxide is mixed with PHY. In one embodiment, the pH of the PHY used in the mixture can range from about 4.5 to about 7.5. In one embodiment, the weight ratio of iron oxide to PHY in the mixture is in a range greater than 0 to about 100:10. In one embodiment, the weight ratio of iron oxide to PHY in the mixture is about 96:4.

In one embodiment, the PHY-treated iron-based colorants is mixed with a solvent before including the remaining components of the hair cosmetic composition. In one embodiment, the solvent may be in liquid form and the PHY-treated iron-based colorants may be in powder form. In one embodiment, an ultra-precision colloid grinding equipment is used to grind the mixture about 3 to 5 times to evenly blend the PHY-treated iron-based colorant with the solvent. In one embodiment, any ultra-precision colloid grinding machine used in the art may be used. In one embodiment, the ultra-precision colloid grinding machine includes Brand Wenzhou MATLONG Machinery Co.,Ltd., Model Number: JMF-100 (specification: 5.5 kw-7.5 kw; motor speed: 290 r/min; working fineness: 2-40 μm). The grinding of the mixture can increase the contact surface area between the PHY-treated iron-based colorant powder and the solvent liquid, which can improve the dispersion of the PHY-treated iron-based colorant powder in the solvent liquid. In one embodiment, the amount of the PHY-treated iron-based colorant ranges from about 159 g to about 232 g after grinding the mixture.

### DETAILED DESCRIPTION OF EXEMPLARY EMBODIMENTS

Embodiments are now discussed in more detail. It is to be understood that the disclosed embodiments are merely illustrative of the disclosure that can be embodied in various forms. In addition, each of the examples given in connection with the various embodiments is intended to be illustrative, and not restrictive. Therefore, specific structural and functional details disclosed herein are not to be interpreted as limiting, but merely as a representative basis for teaching one skilled in the art to variously employ the disclosed embodiments.

Subject matter may be embodied in a variety of different forms and, therefore, covered or claimed subject matter is intended to be construed as not being limited to any example embodiments set forth herein; exemplary embodiments are provided merely to be illustrative. Among other things, for example, subject matter may be embodied as methods, devices, components, or systems. The following detailed description is, therefore, not intended to be taken in a limiting sense.

Throughout the specification and/or claims, terms may have nuanced meanings suggested or implied in context beyond an explicitly stated meaning. Likewise, the phrase “in one embodiment” as used herein does not necessarily refer to the same embodiment, and the phrases “in another embodiment” and “other embodiments” as used herein do not necessarily refer to a different embodiment. It is intended, for example, that covered or claimed subject matter include combinations of exemplary embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

In hair cosmetic products, iron-based colorants, for example, iron oxides, can be used to color or tint hair. Iron oxides can also be found in other cosmetics, including, eye shadows, blushes, powders, lipstick, and mineral makeup. In other hair cosmetic products, an adhesive agent can be used for attaching various hair accessories such as wigs and hair extensions. For example, wigs or hair extensions may have a lace portion that may be attached to a forehead area of a user of the hair product with adhesive properties. The color of lace may not match the color of the wig, the hair, or the skin of the user and may be noticeable. Accordingly, it is desirable for the hair adhesive product to also have hair coloring properties so that it can also color the lace to blend in with the color of the wig, hair, or skin and become inconspicuous.

However, adhesive hair cosmetic compositions that contain iron oxides are inherently unstable. Iron oxides are typically in powder form and may be insoluble in the adhesive hair cosmetic composition because the iron oxides can be separated from the solvent and adhesive components in the composition. Accordingly, the iron oxide may sediment and may not be evenly distributed throughout the composition. Without an even distribution of iron oxide, the adhesive hair cosmetic product may not evenly color hair or lace when applied.

The inventor herein has discovered, surprisingly and unexpectedly, a hair cosmetic composition containing a stable mixture of the iron oxides and adhesive components and a process for manufacturing the composition. The inventor herein has discovered, surprisingly and unexpectedly, that grinding a mixture of propylene glycol and the iron oxides before adding the remaining components of the hair cosmetic composition can increase the contact surface area between the iron oxide and the propylene glycol, and improve the solubility of the iron oxides. The inventor herein has discovered, surprisingly and unexpectedly, that treating a surface of the iron oxides with phytic acid before grinding the mixture of iron oxides and propylene glycol can further improve the solubility of the iron oxides in hair cosmetic composition containing adhesive components.

In one embodiment, the hair cosmetic composition includes iron-based colorants, a solvent, adhesive components, and a propellent. In one embodiment, hair cosmetic composition is formed by placing the iron-based colorants, the solvent, the adhesive components, and the propellant into a pressure container to form an aerosol composition using a process generally understood in the art. In one embodiment, the propellant includes hydrofluorocarbon (HFC) 152a, HFC 134a, and dimethyl ether.

In one embodiment, the iron-based colorants include iron oxides. In one embodiment, the iron-based colorants may be in powder form. In one embodiment, the amount of the iron oxides included in the hair cosmetic composition ranges from about 0.5% (w/w) to about 3.5% (w/w) relative to the overall weight of the cosmetic composition.

In one embodiment, the solvent includes alcohol, water, or a combination thereof. In one embodiment, the solvent includes a polyhydric alcohol, which is almost colorless, odorless, non-greasy, non-volatile and completely soluble in water. In one embodiment, the solvent includes propylene glycol. In one embodiment, the amount of propylene glycol included in the hair cosmetic composition ranges from about 0.5% (w/w) to about 3.5% (w/w) relative to the overall weight of the hair cosmetic composition.

In one embodiment, the adhesive components include AMP-Acrylatescopolymer. In one embodiment, the amount of the adhesive components (e.g., AMP-Acrylatescopolymer) included in the hair cosmetic composition ranges from about 10% (w/w) to 20% (w/w) relative to the overall weight of the cosmetic composition.

In one embodiment, the surface of the iron-based colorant is treated with phytic acid (PHY) to form a PHY-treated iron-based colorant. The iron-based colorant is treated with PHY to form a chelate, so that the surface of the toner has multiple hydrophilic hydroxyl groups (—OH). The multiple hydroxyl groups allow the iron-based colorant to disperse in a liquid phase more easily (e.g., a water phase). PHY is a natural hydrophilic surface treatment agent, which is suitable for water-based makeup and skin care products. The PHY surface treatment allows the iron-based colorant to have high toner loading capacity for easy toning and that the hydrophilicity and viscosity are not affected by pH. In one embodiment, the iron oxide is mixed with PHY. In one embodiment, the pH of the PHY used in the mixture can range from about 4.5 to about 7.5. In one embodiment, the weight ratio of iron oxide to PHY in the mixture is in a range greater than 0 to about 100:10. In one embodiment, the weight ratio of iron oxide to PHY in the mixture is about 96:4.

In one embodiment, the PHY-treated iron-based colorants is mixed with a solvent before including the remaining components of the hair cosmetic composition. In one embodiment, the solvent may be in liquid form and the PHY-treated iron-based colorants may be in powder form. In one embodiment, an ultra-precision colloid grinding equipment is used to grind the mixture about 3 to 5 times to evenly blend the PHY-treated iron-based colorant with the solvent. In one embodiment, any ultra-precision colloid grinding machine used in the art may be used. In one embodiment, the ultra-precision colloid grinding machine includes Brand Wenzhou MATLONG Machinery Co.,Ltd., Model Number: JMF-100 (specification: 5.5 kw-7.5 kw; motor speed: 290 r/min; working fineness: 2-40 μm). The grinding of the mixture can increase the contact surface area between the PHY-treated iron-based colorant powder and the solvent liquid, which can improve the dispersion of the PHY-treated iron-based colorant powder in the solvent liquid. In one embodiment, the amount of the PHY-treated iron-based colorant ranges from about 159 g to about 232 g.

In one embodiment, the hair cosmetic composition is configured for light warm brown hair coloring. In one embodiment, the light warm brown hair cosmetic composition includes ethyl alcohol, dimethyl ether, hydrofluorocarbon 152a, amp-acrylates copolymer, aqua, fragrance, hydrolyzed keratin, panthenol, ci 77492, propylene glycol, ci 77491, ci 77499.

In one embodiment, the hair cosmetic composition is configured for medium brown hair coloring. In one embodiment, the medium brown hair cosmetic composition includes ethyl alcohol, dimethyl ether, hydrofluorocarbon 152a, amp-acrylates copolymer, aqua, fragrance, hydrolyzed keratin, panthenol, ci 77492, propylene glycol, ci 77491, ci 77499.

In one embodiment, the hair cosmetic composition is configured for medium dark brown hair coloring. In one embodiment, the medium dark brown hair cosmetic composition includes ethyl alcohol, dimethyl ether, hydrofluorocarbon 152a, amp-acrylates copolymer, aqua, fragrance, hydrolyzed keratin, panthenol, ci 77492, propylene glycol, ci 77491, ci 77499.

In one embodiment, the hair cosmetic composition is configured for dark brown hair coloring. In one embodiment, the dark brown hair cosmetic composition includes ethyl alcohol, dimethyl ether, hydrofluorocarbon 152a, amp-acrylates copolymer, aqua, fragrance, hydrolyzed keratin, panthenol, ci 77492, propylene glycol, ci 77491, ci 77499.

It will be understood by those having ordinary skill in the art and possession of the present disclosure that the embodiments described herein are merely exemplary in nature and that a person skilled in the art may make many variations and modifications thereto without departing from the scope of the present invention. All such variations and modifications are intended to be included within the scope of the invention.

What is claimed is:

1. A method for preparing a hair cosmetic composition, the method comprising the steps of: treating a colorant with a phytic acid; preparing a mixture including the colorant treated with phytic acid, and a solvent; grinding the mixture about 3 to 5 times using an ultra-precision colloid grinding machine; adding an adhesive component to the mixture; and adding a propellant to the mixture.

2. The method of claim 1, wherein the colorant includes an iron oxide.

3. The method of claim 2, wherein an amount of the iron oxide is in a range from about 0.5% (w/w) to about 3.5% (w/w) relative to the overall weight of the cosmetic composition.

4. The method of claim 2, wherein a weight ratio of the iron oxide to the phytic acid used in the treating of the colorant with phytic acid is in a range greater than 0 to about 100:10.

5. The method of claim 4, wherein the weight ratio of the iron oxide to the phytic acid used in the treating of the colorant with phytic acid is about 96:4.

6. The method of claim 1, wherein a pH of the phytic acid is in a range from about 4.5 to about 7.5.

7. The method of claim 1, wherein the solvent includes propylene glycol.

8. The method of claim 7, wherein an amount of the propylene glycol in the hair cosmetic composition is in a range from about 0.5% (w/w) to about 3.5% (w/w) relative to the overall weight of the hair cosmetic composition.

9. The method of claim 1, wherein the adhesive component includes AMP-Acrylatescopolymer.

10. The method of claim 9, wherein an amount of the AMP-Acrylatescopolymer in the hair cosmetic composition is in a range from about 10% (w/w) to 20% (w/w) relative to the overall weight of the cosmetic composition.

11. A hair cosmetic composition used for attaching hair accessories, comprising a colorant treated with phytic acid to create additional hydroxyl groups on a surface of the colorant, a solvent, and an adhesive component, wherein said colorant treated with phytic acid is configured to evenly disperse in said solvent before adding said adhesive component.

12. The hair cosmetic composition of claim 11, wherein said colorant includes an iron oxide.

13. The hair cosmetic composition of claim 12, wherein an amount of said iron oxide is in a range from about 0.5% (w/w) to about 3.5% (w/w) relative to the overall weight of the cosmetic composition.

14. The hair cosmetic composition of claim 12, wherein a weight ratio of said iron oxide to said phytic acid used in the treating of the colorant with phytic acid is in a range greater than 0 to about 100:10.

15. The hair cosmetic composition of claim 14, wherein a weight ratio of said iron oxide to said phytic acid used in the treating of the colorant with phytic acid is about 96:4.

16. The hair cosmetic composition of claim 11, wherein a pH of said phytic acid is in a range from about 4.5 to about 7.5.

17. The hair cosmetic composition of claim 11, wherein said solvent includes propylene glycol.

18. The hair cosmetic composition of claim 17, wherein an amount of said propylene glycol in the hair cosmetic composition is in a range from about 0.5% (w/w) to about 3.5% (w/w) relative to the overall weight of the hair cosmetic composition.

19. The hair cosmetic composition of claim 11, wherein said adhesive component includes AMP-Acrylatescopolymer.

20. The hair cosmetic composition of claim 19, wherein an amount of said AMP-Acrylatescopolymer in the hair cosmetic composition is in a range from about 10% (w/w) to 20% (w/w) relative to the overall weight of the cosmetic composition.

21. The hair cosmetic composition of claim 11, where said colorant treated with phytic acid is prepared in a mixture, and the mixture is ground about 3 to 5 times using an ultra-precision colloid grinding machine.

22. A hair cosmetic composition, comprising ethyl alcohol, dimethyl ether, hydrofluorocarbon 152a, amp-acrylates copolymer, aqua, fragrance, hydrolyzed keratin, panthenol, ci 77492, propylene glycol, ci 77491, ci 77499.
